(19) United States
(12) Patent Application Publication
(10) Pub. No.: US 2003/0003057 A1
(43) Pub. Date: Jan. 2, 2003

(54) Methods for administering leuprolide by inhalation

(76) Inventors: Jeffry Weers, Half Moon Bay, CA (US); Thomas E. Tarara, Burlingame, CA (US)

Correspondence Address: INHALE THERAPEUTIC SYSTEMS, INC, 150 INDUSTRIAL ROAD, SAN CARLOS, CA, 94070, US

(21) Appl. No.: 10/141,219

(22) Filed: May 7, 2002

Related U.S. Application Data
(63) Continuation-in-part of application No. 09/851,226, filed on May 8, 2001.
(63) Continuation-in-part of application No. 09/888,311, filed on Jun. 22, 2001.
(60) Provisional application No. 60/216,621, filed on Jul. 7, 2000.
(60) Provisional application No. 60/216,621, filed on Jul. 7, 2000.

(51) Int. Cl. A61L 9/04; A61K 9/14

(52) U.S. Cl. 424/46

(57) ABSTRACT

Phospholipid based particulate compositions of leuprolide and methods of pulmonary administration via dry powder inhalers are provided. The leuprolide particulate compositions are particularly suited to the treatment of diseases and disorders associated with elevated or inappropriate levels of sex-hormone or that benefit from inhibition of gonadotropin secretion, such as prostate cancer, endometriosis, and central precocious puberty. The leuprolide compositions for inhalation are engineered to be highly dispersible and provide rapid absorption of the active agent so delivered, as well as to exhibit substantially independent emitted doses and lung deposition as functions of device resistance and inspiratory flow rates, respectively.

### RELATED APPLICATIONS

This application is a continuation-in-part of U.S. patent application Ser. No. 09/851,226, filed May 8, 2001 and a continuation-in-part of U.S. patent application Ser. No. 09/888,311, filed Jun. 22, 2001, the contents of which are incorporated by reference herein. U.S. patent application Ser. Nos. 09/851,226 and 09/888,311, in turn claim the priority of U.S. Provisional Application U.S. Provisional Application 60/216,621 filed Jul. 7, 2000.

### FIELD OF THE INVENTION

The present invention relates to particulate compositions of leuprolide and methods for inhalation delivery thereof, particularly for the treatment of prostate cancer, endometriosis, central precocious puberty and other diseases and disorders that benefit from inhibition of gonadotropin secretion. In particular, the present invention provides particulate compositions of leuprolide and methods for pulmonary administration via dry powder inhalers.

### BACKGROUND OF THE INVENTION

This invention relates generally to the field of drug delivery, and in particular to the delivery of pharmaceutical formulations of leuprolide to the lungs to treat diseases and disorders that benefit from the inhibition of gonadotropin secretion. More specifically, the invention relates to the aerosolization of pharmaceutical formulations of leuprolide, preferably using energy created by patient inhalation.

Pulmonary delivery techniques rely on the inhalation of a pharmaceutical formulation by the patient so that the active drug within the dispersion can reach the distal (alveolar) regions of the lung. A variety of aerosolization systems have been proposed to disperse pharmaceutical formulations. For example, U.S. Pat. Nos. 5,785,049 and 5,740,794, the disclosures of which are herein incorporated by reference, describe exemplary active powder dispersion devices which utilize a compressed gas to aerosolize a powder.

Other types of aerosolization systems include MDI's (which typically have a drug that is stored in a propellant), nebulizers (which aerosolize liquids using compressed gas, usually air), and the like. The administration of leuprolide aerosols via MDIs is disclosed in U.S. Pat. Nos. 4,851,211, 5,853,740, 5,635,159, 5,635,161, and 5,676,931.

Another aerosolization technique is the use of inspired gases to disperse the pharmaceutical formulation. In this way, the patient is able to provide the energy needed to aerosolize the formulation by the patient's own inhalation. This insures that aerosol generation and inhalation are properly synchronized. Utilization of the patient's inspired gases can be challenging in several respects. For example, for some pharmaceutical formulations, such as insulin, it may be desirable to limit the inhalation flow rate within certain limits. For example, PCT/US99/04654, filed Mar. 11, 1999, provides for the pulmonary delivery of insulin at rates less than 17 liters per minute. As another example, copending U.S. patent application Ser. No. 09/414,384 describes pulmonary delivery techniques where a high flow resistance is provided for an initial period followed by a period of lower flow resistance. The complete disclosures of all the above references are herein incorporated by reference.

Another challenge in utilizing the patient's inspired gases is that the inspiration flow rate can drastically vary between individuals. For all commercially available dry powder inhalers, aerosolization and dispersion of the drug formulation are dependent on the inspiratory effort of the patient in inhaling a dose. This effort produces an air flow rate through the device which is governed by the inherent resistance of the device. Variability in inspiratory effort may affect the ability of the formulation to be dispersed within a gas stream, the ability to deagglomerate a powdered formulation, and/or the ability of the aerosolized formulation to adequately reach the deep lung.

Problems associated with variability among patient inspiratory efforts have been addressed through modifications of dry powder inhaler device designs. For example, WO 01/00263 and WO 00/21594, hereby incorporated in their entirety by reference, disclose dry powder inhalers (DPI) including flow regulation and flow resistance modulation. Examples of other DPIs are disclosed in U.S. Pat. Nos. 4,995,385, 6,230,707, 6,032,666, 5,873,360, 4,524,769, 5,577,497 and 5,727,546, and PCT applications WO 99/45986, WO 99/45987, and WO 97/25086, herein incorporated in their entirety by reference.

Due to its spreading characteristics on lung epithelia, surfactant has been proposed as the ideal carrier for delivery of drugs to the lung, and via the lung to the systemic circulation. Once again, achieving efficient delivery to the lung is important, especially in light of the potential high cost of many of the current products. One potential way to deliver drugs in phospholipids is as a dry powder aerosolized to the lung. Most fine powders (<5 μm) exhibit poor dispersibility. This can be problematic when attempting to deliver, aerosolize, and/or package the powders.

Examples of particulate compositions incorporating a surfactant are disclosed in PCT publications WO 99/16419, WO 99/38493, WO 99/66903, WO 00/10541, and U.S. Pat. No. 5,855,913, which are hereby incorporated in their entirety by reference. Of particular interest to the present invention are recent particle engineering technologies, such as the PulmoSphere® process (Inhale Therapeutic Systems, San Carlos, Calif.), that have led to more easily dispersible powders with improved aerodynamic properties. These powders have been shown to deliver drugs efficiently to the lower respiratory tract using relatively simple and inexpensive dry powder inhalers. U.S. disclosed, for example, in co-pending U.S. patent application Ser. No. 09/888,311, filed Jun. 22, 2001, incorporated by reference herein.

### SUMMARY OF THE INVENTION

In contrast to the prior art emphasis on device design to address issues commonly associated with patient variability in inspiratory effort, the present invention is directed to a particle engineering approach to overcome such issues. It has surprisingly been found that the leuprolide particles of the present invention when administered from a simple passive DPI result in an emitted dose and lung deposition that is substantially independent of device resistance and inspiratory effort, respectively.

Thus, the present invention provides for dry powder compositions of leuprolide and methods of inhalation leuprolide therapy. According to a preferred embodiment, the particulate leuprolide compositions are efficiently delivered to the deep lung. Leuprolide particles may be delivered alone, as a single agent formulation, or in combination with other active agents and/or excipients. Phospholipids are a preferred excipient.

According to one embodiment, the compositions of the present invention may be delivered from a simple passive DPI device. The present compositions allow for more efficient delivery to the lung.

It is a further object of the present invention that the improvements in dispersibility obtained by the present leuprolide particulate compositions allow for a simple, passive inhaler device to be utilized, in spite of the fact that particles less than 5 μm are contemplated and generally preferred. Present state-of-the-art formulations for fine particles utilize blends with large lactose particles to improve dispersibility. When placed in a passive DPI device such formulations exhibit a strong dependence of emitted dose and lung deposition on the patient's inspiratory flowrate. The present compositions exhibit little flowrate dependence on the emitted dose and lung deposition.

These and other objects and features of the invention will become more fully apparent when the following detailed description is read in conjunction with the accompanying figures and examples.

### BRIEF DESCRIPTION OF THE DRAWINGS

FIG. 1 depicts a Scanning electron micrograph of the leuprolide acetate particles manufactured using a phospholipid emulsion based spray-drying process described herein.

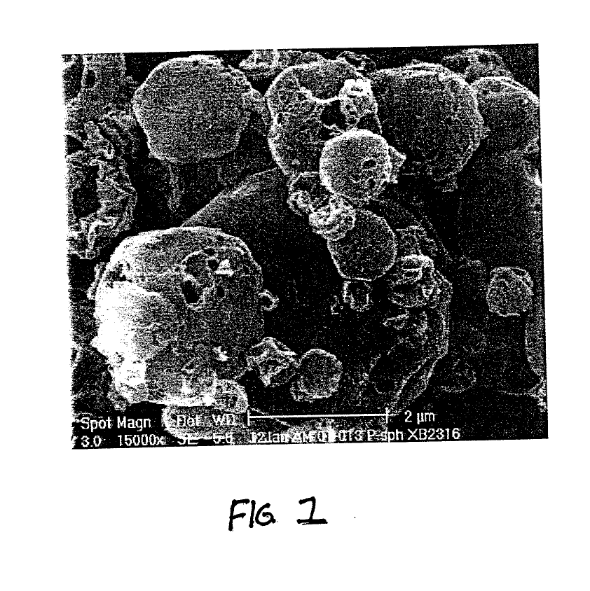

FIG. 2 depicts the pharmokinetic and bioavailability profiles of particulate compositions of leuprolide acetate administered according to the present invention as compared to those of leuprolide acetate administered intravenously.

### DEFINITIONS

The present invention is specifically directed to particulate compositions that include leuprolide as the active agent. The term “leuprolide” as used herein encompasses not only the base form of the drug but pharmaceutical salts thereof, such as leuprolide acetate. Leuprolide is a synthetic nonapeptide analog of naturally occurring gonadotropin releasing hormone (GnRH) and acts as an agonist of leutenizing hormone releasing hormone (LH-RH). The analog possesses greater potency than the natural hormone. When given continuously and in therapeutic doses, leuprolide acts as a potent inhibitor of gonadotropin secretion, which ultimately leads to reduced circulating levels of gonadal steroids, such as testosterone and estrone. This effect is generally reversible upon discontinuation of drug therapy.

Administration of leuprolide has been shown to result in the inhibition of growth of certain hormone dependent tumors, such as prostatic tumors in males and mammary tumors in females, as well as the atrophy of the reproductive organs. Accordingly, it finds particular utility in the treatment diseases and disorders associated with elevated or inappropriate levels of sex-hormone such as prostate cancer, endometriosis, central precocious puberty, Pharmaceutically formulated leuprolide acetate is commercially available from TAP Pharmaceuticals (Lake Forest, Ill.) under the tradenames LUPRON® and LUPRON DEPOT®. Leuprolide may be administered alone or in combination with one or more additional active agents.

As used herein, the term “emitted dose” or “ED” refers to an indication of the delivery of dry powder from a suitable inhaler device after a dispersion event from a powder unit or reservoir. ED is defined as the ratio of the dose delivered by an inhaler device (described in detail below) to the nominal dose (i.e., the mass of powder per unit dose placed into a suitable inhaler device prior to dispersion). The ED is an experimentally-determined amount, and is typically determined using an in-vitro device set up which mimics patient dosing. To determine an ED value, a nominal dose of dry powder (as defined above) is placed into a suitable dry powder inhaler, which is then actuated, dispersing the powder. The resulting aerosol cloud is then drawn by vacuum from the device, where it is captured on a tared filter attached to the device mouthpiece. The amount of powder that reaches the filter constitutes the emitted dose. For example, for a 5 mg, dry powder-containing blister pack placed into an inhalation device, if dispersion of the powder results in the recovery of 4 mg of powder on a tared filter as described above, then the ED for the dry powder composition is: 4 mg (delivered dose)/5 mg (nominal dose)×100=80%.

“Mass median diameter” or “MMD” is a measure of particle size, since the powders of the invention are generally polydisperse (i.e., consist of a range of particle sizes). MMD values as reported herein are determined by laser diffraction.

“Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, generally in air, as the particle. The aerodynamic diameter encompasses particle shape, density and physical size of a particle. Techniques for measuring MMAD include Multistage Liquid Impinger as known in the art.

As used herein, “passive dry powder inhaler” refers to an inhalation device which relies upon the patient's inspiratory effort to disperse and aerosolize a drug formulation contained within the device and does not include inhaler devices which comprise a means for providing energy to disperse and aerosolize the drug formulation, such as pressurized gas and vibrating or rotating elements.

### DETAILED DESCRIPTION OF THE INVENTION

The present invention is directed to methods for the pulmonary administration of leuprolide to the respiratory tract for local or systemic therapy via aerosolization. The invention is based, at least in part, on the surprising discovery of the beneficial aerosolization of phospholipid-containing particulate leuprolide compositions. According to the present invention, inhalation of leuprolide via a passive dry powder inhaler is effective to achieve peak serum concentration of leuprolide in blood of at least 3.0 μg/ml, preferably at least 4.0 μg/ml, and most preferably 5.0 μg/ml. Maximum serum levels are attained within 90 minutes, preferably 80 minutes, and most preferably within 70 minutes. Administration according to the invention results in an area under the concentration-time curve (AUC—24 hr) of at least 10 μg-hr/ml, preferably 15 μg-hr/ml and exhibits a bioavailability of about 18% with respect to intravenous administration.

In a broad sense, phospholipids suitable for use in the present invention include any of those known in the art. According to a preferred embodiment, the phospholipid is most preferably a saturated phospholipid. According to a particularly preferred embodiment, saturated phosphatidylcholines are used as the phospholipid of the present invention. Preferred acyl chain lengths are 16:0 and 18:0 (i.e. palmitoyl and stearoyl). Leoprolide loading can vary between about 0.1% and 90% w/w, preferably 2-80% w/w, most preferably 5-50% w/w.

Phospholipids from both natural and synthetic sources are compatible with the present invention and may be used in varying concentrations to form the structural matrix. Generally compatible phospholipids comprise those that have a gel to liquid crystal phase transition greater than about 40° C. Preferably the incorporated phospholipids are relatively long chain (i.e. C₁₆-C₂₂) saturated lipids and more preferably comprise saturated phospholipids, most preferably saturated phosphatidylcholines having acyl chain lengths of 16:0 or 18:0 (palmitoyl and stearoyl). Exemplary phospholipids useful in the disclosed stabilized preparations comprise, phosphoglycerides such as dipalmitoylphosphatidylcholine, disteroylphosphatidylcholine, diarachidoylphosphatidylcholine dibehenoylphosphatidylcholine, diphosphatidyl glycerol, short-chain phosphatidylcholines, long-chain saturated phosphatidylethanolamines, long-chain saturated phosphatidylserines, long-chain saturated phosphatidylglycerols, long-chain saturated phosphatidylinositols.

In addition to the phospholipid, a co-surfactant or combinations of surfactants, including the use of one or more in the liquid phase and one or more associated with the particulate compositions are contemplated as being within the scope of the invention. By “associated with or comprise” it is meant that the particulate compositions may incorporate, adsorb, absorb, be coated with or be formed by the surfactant. Surfactants include fluorinated and nonfluorinated compounds and are selected from the group consisting of saturated and unsaturated lipids, nonionic detergents, nonionic block copolymers, ionic surfactants and combinations thereof. In those embodiments comprising stabilized dispersions, such nonfluorinated surfactants will preferably be relatively insoluble in the suspension medium. It should be emphasized that, in addition to the aforementioned surfactants, suitable fluorinated surfactants are compatible with the teachings herein and may be used to provide the desired preparations.

Compatible nonionic detergents suitable as co-surfactants comprise: sorbitan esters including sorbitan trioleate (Span™ 85), sorbitan sesquioleate, sorbitan monooleate, sorbitan monolaurate, polyoxyethylene (20) sorbitan monolaurate, and polyoxyethylene (20) sorbitan monooleate, oleyl polyoxyethylene (2) ether, stearyl polyoxyethylene (2) ether, lauryl polyoxyethylene (4) ether, glycerol esters, and sucrose esters. Other suitable nonionic detergents can be easily identified using McCutcheon's Emulsifiers and Detergents (McPublishing Co., Glen Rock, N.J.) which is incorporated herein in its entirety. Preferred block copolymers include diblock and triblock copolymers of polyoxyethylene and polyoxypropylene, including poloxamer 188 (Pluronic™ F-68), poloxamer 407 (Pluronic™ F-127), and poloxamer 338. Ionic surfactants such as sodium sulfosuccinate, and fatty acid soaps may also be utilized.

Other lipids including glycolipids, ganglioside GM1, sphingomyelin, phosphatidic acid, cardiolipin; lipids bearing polymer chains such as polyethylene glycol, chitin, hyaluronic acid, or polyvinylpyrrolidone; lipids bearing sulfonated mono-, di-, and polysaccharides; fatty acids such as palmitic acid, stearic acid, and oleic acid; cholesterol, cholesterol esters, and cholesterol hemisuccinate may also be used in accordance with the teachings of this invention.

The microparticles of the present invention may also include a biocompatible, preferably biodegradable polymer, copolymer, or blend or other combination thereof. In this respect useful polymers comprise polylactides, polylactide-glycolides, cyclodextrins, polyacrylates, methylcellulose, carboxymethylcellulose, polyvinyl alcohols, polyanhydrides, polylactams, polyvinyl pyrrolidones, polysaccharides (dextrans, starches, chitin, chitosan, etc.), hyaluronic acid, proteins, (albumin, collagen, gelatin, etc.). Examples of polymeric resins that would be useful for the preparation of perforated ink microparticles include: styrene-butadiene, styrene-isoprene, styrene-acrylonitrile, ethylene-vinyl acetate, ethylene-acrylate, ethylene-acrylic acid, ethylene-methylacrylatate, ethylene-ethyl acrylate, vinyl-methyl methacrylate, acrylic acid-methyl methacrylate, and vinyl chloride-vinyl acetate. Those skilled in the art will appreciate that, by selecting the appropriate polymers, the delivery efficiency of the particulate compositions and/or the stability of the dispersions may be tailored to optimize the effectiveness of the active or agent.

Besides the aforementioned polymer materials and surfactants, it may be desirable to add other excipients to a particulate composition to improve particle rigidity, production yield, emitted dose and deposition, shelf-life and patient acceptance. Such optional excipients include, but are not limited to: coloring agents, taste masking agents, buffers, hygroscopic agents, antioxidants, and chemical stabilizers. Further, various excipients may be incorporated in, or added to, the particulate matrix to provide structure and form to the particulate compositions (i.e. microspheres such as latex particles). In this regard it will be appreciated that the rigidifying components can be removed using a post-production technique such as selective solvent extraction.

Other excipients may include, but are not limited to, carbohydrates including monosaccharides, disaccharides and polysaccharides. For example, monosaccharides such as dextrose (anhydrous and monohydrate), galactose, mannitol, D-mannose, sorbitol, sorbose and the like; disaccharides such as lactose, maltose, sucrose, trehalose, and the like; trisaccharides such as raffinose and the like; and other carbohydrates such as starches (hydroxyethylstarch), cyclodextrins and maltodextrins. Other excipients suitable for use with the present invention, including amino acids, are known in the art such as those disclosed in WO 95/31479, WO 96/32096, and WO 96/32149, hereby incorporated in their entirety by reference. Mixtures of carbohydrates and amino acids are further held to be within the scope of the present invention. The inclusion of both inorganic (e.g. sodium chloride, etc.), organic acids and their salts (e.g. carboxylic acids and their salts such as sodium citrate, sodium ascorbate, magnesium gluconate, sodium gluconate, tromethamine hydrochloride, etc.) and buffers is also contemplated. The inclusion of salts and organic solids such as ammonium carbonate, ammonium acetate, ammonium chloride or camphor are also contemplated.

According to a preferred embodiment, a metal cation, preferably calcium is added to the feed stock from which the particles are prepared as disclosed in WO 01/85136 and WO 01/85137, hereby incorporated in their entirety by reference.

Yet other preferred embodiments include particulate compositions that may comprise, or may be coated with, charged species that prolong residence time at the point of contact or enhance penetration through mucosae. For example, anionic charges are known to favor mucoadhesion while cationic charges may be used to associate the formed microparticulate with negatively charged bioactive agents such as genetic material. The charges may be imparted through the association or incorporation of polyanionic or polycationic materials such as polyacrylic acids, polylysine, polylactic acid and chitosan.

The medicament is formulated in a way such that it readily disperses into discrete particles with an MMD between 0.5 to 20 μm, preferably 0.5-5 μm, and are further characterized by an aerosol particle size less than about 10 μm mass median aerodynamic diameter (MMAD), and preferably less than 5.0 μm. The mass median aerodynamic diameters of the powders will characteristically range from about 0.5-10 μm, preferably from about 0.5-5.0 μm MMAD, more preferably from about 1.0-4.0 μm MMAD.

The administration methods of the present invention utilize passive DPIs. Examples of passive DPIs suitable for administration of the particulate compositions of the present invention are disclosed in U.S. Pat. Nos. 5,673,686, and 4,995,385 and PCT application nos. WO 00/72904, WO 00/21594, and WO 01/00263, hereby incorporated in their entirety by reference. DPI formulations are typically packaged in single dose units such as those disclosed in the above mentioned patents or they employ reservoir systems capable of metering multiple doses with manual transfer of the dose to the device.

Particularly preferred embodiments of the invention incorporate spray dried, hollow and porous particulate compositions as disclosed in WO 99/16419, hereby incorporated in its entirety by reference. Such particulate compositions comprise particles having a relatively thin porous wall defining a large internal void, although, other void containing or perforated structures are contemplated as well.

Compositions according to the present invention typically yield powders with bulk densities less than 0.5 g/cm³ or 0.3 g/cm³, preferably less 0.1 g/cm³ and most preferably less than 0.05 g/cm³. By providing particles with very low bulk density, the minimum powder mass that can be filled into a unit dose container is reduced, which eliminates the need for carrier particles. That is, the relatively low density of the powders of the present invention provides for the reproducible administration of relatively low dose pharmaceutical compounds. Moreover, the elimination of carrier particles will potentially minimize throat deposition and any “gag” effect, since the large lactose particles will impact the throat and upper airways due to their size.

It will be appreciated that the particulate leuprolide compositions disclosed herein comprise a structural matrix that exhibits, defines or comprises voids, pores, defects, hollows, spaces, interstitial spaces, apertures, perforations or holes. The absolute shape (as opposed to the morphology) of the particulate microstructure is generally not critical and any overall configuration that provides the desired characteristics is contemplated as being within the scope of the invention. Accordingly, preferred embodiments can comprise approximately microspherical shapes. However, collapsed, deformed or fractured particulates are also compatible.

In accordance with the teachings herein the particulate compositions will preferably be provided in a “dry” state. That is the microparticles will possess a moisture content that allows the powder to remain chemically and physically stable during storage at ambient temperature and easily dispersible. As such, the moisture content of the microparticles is typically less than 20% by weight, and preferably less than 10% by weight. In some instances the moisture content will be as low as 1% by weight. Of course it will be appreciated that the moisture content is, at least in part, dictated by the formulation and is controlled by the process conditions employed, e.g., inlet temperature, feed concentration, pump rate, and blowing agent type, concentration and post drying.

Reduction in bound water leads to significant improvements in the dispersibility and flowability of phospholipid based powders, leading to the potential for highly efficient delivery of powdered lung surfactants or particulate composition comprising active agent dispersed in the phospholipid. The improved dispersibility allows simple passive DPI devices to be used to effectively deliver these powders.

As seen from the passages above, various components may be associated with, or incorporated in the particulate compositions of the present invention. Similarly, several techniques may be used to provide particulates having the desired morphology (e.g. a perforated or hollow/porous configuration), dispersibility and density. Among other methods, particulate compositions compatible with the instant invention may be formed by techniques including spray drying, vacuum drying, solvent extraction, emulsification or lyophilization, and combinations thereof. It will further be appreciated that the basic concepts of many of these techniques are well known in the prior art and would not, in view of the teachings herein, require undue experimentation to adapt them so as to provide the desired particulate compositions.

While several procedures are generally compatible with the present invention, particularly preferred embodiments typically comprise particulate compositions formed by spray drying. As is well known, spray drying is a one-step process that converts a liquid feed to a dried particulate form. With respect to pharmaceutical applications, it will be appreciated that spray drying has been used to provide powdered material for various administrative routes including inhalation. See, for example, M. Sacchetti and M. M. Van Oort in: Inhalation Aerosols: Physical and Biological Basis for Therapy, A. J. Hickey, ed. Marcel Dekkar, New York, 1996, which is incorporated herein by reference. Examples of spray drying methods and systems suitable for making the dry powders of the present invention are disclosed in WO 99/16419 previously incorporated by reference and in U.S. Pat. Nos. 6,077,543, 6,051,256, 6,001,336, 5,985,248, and 5,976,574, hereby incorporated in their entirety by reference.

Whatever production method is ultimately selected for production of the particulate compositions, the resulting powders have a number of advantageous properties that make them particularly compatible for use in devices for inhalation therapies. In particular, the physical characteristics of the particulate compositions make them extremely effective for use in passive dry powder inhalers. As such, the particulate compositions provide for the effective pulmonary administration of active agents.

In especially preferred embodiments the particulate compositions will comprise a powder of dry, hollow, porous microspherical shells of approximately 1 to 10 μm or 1 to 5 μm MMD, with shell thicknesses of approximately 0.1 μm to approximately 0.5 μm. It is a particular advantage of the present invention that the particulate concentration of the dispersions and structural matrix components can be adjusted to optimize the delivery characteristics of the selected particle size.

The foregoing description will be more fully understood with reference to the following Examples. Such Examples, are, however, merely representative of preferred methods of practicing the present invention and should not be read as limiting the scope of the invention.

### EXAMPLE 1

### Preparation of Spray-Dried Particles of Leuprolide Acetate

A single feed solution was prepared under defined conditions. The feed solution was comprised of leuprolide acetate in the aqueous phase of a fluorocarbon-in-water emulsion. The emulsion composition is listed in Table 1 below. Accordingly, DSPC and calcium chloride dihydrate were dispersed in approximately 400 mL SWFI (T=60-70 C) using an Ultra-Turrax T-50 mixer at 8000 rpm for 2 to 5 minutes. The perflubron was then added drop wise during mixing. After the addition was complete, the emulsion was mixed for an additional period of not less than 5 minutes at 10,000 rpm. The resulting coarse emulsion was then homogenized under high pressure with an Avestin C-5 homogenizer (Ottawa, Canada) at 19,000 psi for 5 discrete passes and then spray dried on a Bucchi spray dryer (BÜchi, Flawil, Switzerland).

TABLE 1
Leuprolide Acetate Emulsion Composition

| Emulsion Components | Amount (grams) | % solids |
|---|---|---|
| DSPC | 7.33 | 73% |
| Calcium Chloride | 0.67 | 7% |
| Perflubron | 200 | NA |
| SWFI | 400 | NA |
| Leuprolide Acetate | 2.00 | 20% |

### Administration of Leuprolide Powders

Inhalation

The leuprolide powder was collected and then filled into size #2 capsules, approximately 5 mg of 20% leuprolide acetate powder per capsule. Capsules were bottled and the bottles individually enclosed in an appropriately labeled sealed foil pouch with desiccant and stored at room temperature.

The leuprolide powders were administered using the Turbospin® dry powder inhaler (PH&T, Italy), loaded with one size #2 capsule filled with drug powder. Subjects were instructed to inhale deeply and forcefully while wearing nose clips. Inhalations were followed by a 5 second breath hold prior to exhalation. Each dose contained approximately 1 mg of leuprolide acetate (5 mg of 20% leuprolide acetate powder), inhaled in a single inhalation.

Intravenous

Leuprolide was supplied in multiple dose vials as a sterile aqueous solution for injection. Each vial contained 2.8 ml of leuprolide acetate 5 mg/ml, sodium chloride for tonicity adjustment, 9 mg/ml of benzyl alcohol as a preservative and water for injection. The pH was adjusted with sodium hydroxide or acetic acid. Prior to administration, each vial was visually inspected for particulate matter or discoloration. The leuprolide vials were stored at room temperature.

Each subject received an intravenous injection of 0.5 mg leuprolide acetate (0.1 ml solution) in the antecubital vein. The injection was given in the opposite arm to that used for blood sample for pharmacokinetic assessments. There were 2 dosing visits, followed by a post study medical 3 to 14 days after the last dose. The interval between doses was no less than 7 days. Venous blood samples were collected in Periods 1 and 2. Blood samples (10 ml) for determination of plasma leuprolide concentrations were drawn predose and 5, 15, 30, 45 minutes, and at 1, 1.5, 2, 4, 6, 8, 12, 16, and 24 hours postdose.

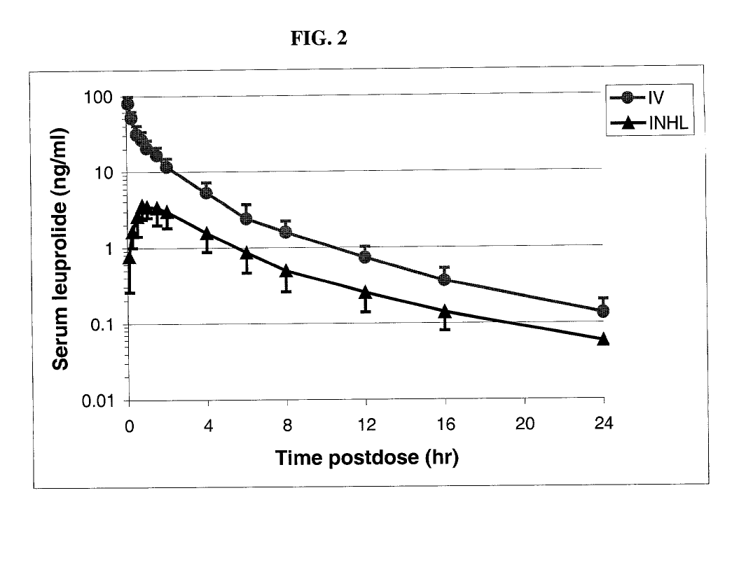

FIG. 2 depicts the pharmacokinetic profiles of the leoprolide administration via inhalation and intravenous injection

Data Analysis

Noncompartmental pharmacokinetic parameters (including Cmax, tmax, AUC, and t½) were calculated from observed serum concentration-time data for each treatment of each subject in accordance with the methods disclosed in Gibaldi M, Perrier F, Pharmacokinetics. 2nd edition revised and expanded, Drugs and the Pharmaceutical Sciences, Volume 15, Marcel Dekker Inc, New York and Basel, 1982 Marcel Dekker Inc, New York, 1988. The results are depicted in Table 2 below.

| | |
|---|---|
| AUC_{(0→24)} | Area under the concentration-time curve |
| | (AUC) from time zero to 24 hr, |
| | calculated by linear trapezoidal method. |
| AUC_{(0→∞)} | Area under the concentration-time curve |
| | (AUC) from time zero to infinity. |
| Cₘₐₓ | Maximum concentration observed postdose. |
| tₘₐₓ | Time at which Cₘₐₓ occurs. |
| t_{½} | Terminal elimination half-life |

Relative bioavailability: The bioavailability (BA) of the leuprolide formulation delivered from Turbospin, relative to intravenous injection of leuprolide, was determined from the ratio of log-transformed AUC values, according to the following equation,

Relative BA=(InAUC_{PulmoSphere}/InAUCᵢᵥ) * (nominal dose_{PulmoSphere}/doseᵢᵥ),

where the nominal dose is the amount of leuprolide acetate loaded into the delivery device, and the iv dose is 0.5 mg . The leoprolide formulation administered via inhalation exhibited a bioavailabiliy of 18% compared to the leoprolite intravenous administration.

TABLE 2
Mean Parameter Value (SD), n = 12

| | Cmax ng/ | | | AUC ng | |
|---|---|---|---|---|---|
| Treatment | ml | Tmax hr | Half-life hr | hr/ml | F % |
| PulmoSphere | 4.0 (1.2) | 1.1 (0.4) | 3.0 (0.4) | 16.0 (6.0) | 17 (3) |
| IV | | | 3.5 (0.4) | 91 (24) | |

The invention has now been described in detail for purposes of clarity and understanding. However, it will be appreciated that certain changes and modifications may be practiced within the scope of the appended claims.

We claim:

1. A method for administering leuprolide to the lungs comprising the steps of:
(i) providing a dry powder composition comprising leuprolide particles having a particle size ranging from 1 to 20 microns, a mass median aerodynamic diameter of less than 5 microns, and a bulk density of less than 0.5 g/cm³;
(ii) loading the composition into a dry powder inhalation device; and
(iii) inhaling the composition from the inhalation device in order to achieve peak leuprolide concentration in serum within 90 minutes of inhalation.

2. A method according to claim 1 wherein the peak leuprolide concentration in serum is achieved within 80 minutes of inhalation.

3. A method according to claim 1 wherein the peak leuprolide concentration in serum is achieved within 70 minutes of inhalation.

4. A method according to claim 1 wherein the device is a passive dry powder inhaler.

5. The method according to claim 4, wherein the emitted dose is greater than 60%.

6. The method according to claim 4, wherein the emitted dose is greater than 80%.

7. The method according to claim 4, wherein the emitted dose is greater than 90%.

8. The method according to claim 1 wherein the Cₘₐₓ is at least 3.0 μg/ml

9. The method according to claim 1, wherein the Cₘₐₓ is at least 4.0 μg/ml.

10. The method according to claim 1, wherein the Cₘₐₓ is at least 5.0 μg/ml.

11. The method according to claim 1, wherein inhaling the composition from the inhaler results in an area under the curve (AUC) dose of at least 10 μg-hr/ml.

12. The method according to claim 1, wherein inhaling the composition from the inhaler results in an area under the curve (AUC) of at least 16 μg-hr/ml.

13. The method according to claim 4, wherein said particles have a mass median aerodynamic diameter of about 5 microns or less.

14. The method according to claim 13, wherein said particles have a mass median diameter of about 0.5-20 microns.

15. The method according to claim 14, wherein said particles have a mass median diameter of less than 5 microns.

16. The method according to claim 1, wherein the relative bioavailability of the leuprolide composition is 18% compared to intravenous administration.

17. A method of administering leuprolide to the lungs comprising the steps of:
(i) providing a dry powder composition comprising particles of leuprolide, said particles having a particle size ranging from 1 to 20 microns, a mass median aerodynamic diameter of less than 5 microns, and a bulk density of less than 0.5 g/cm³;
(ii) loading the composition into a dry powder inhalation device; and
(iii) inhaling the composition from the inhalation device in order to achieve a peak leuprolide concentration in serum (Cₘₐₓ) of at least 3.0 μg/ml.

18. The method of claim 17 wherein the dry powder inhalation device is a passive dry powder inhaler.

19. The method according to claim 18, wherein the emitted dose is greater than 60%.

20. The method according to claim 18, wherein the emitted dose is greater than 80%.
